(19) United States
(12) Patent Application Publication
(10) Pub. No.: US 2021/0145762 A1
(43) Pub. Date: May 20, 2021

(54) METHODS OF TREATING OXIDATIVE STRESS TO MODULATE REDOX BALANCE, PREVENTING OXIDATIVE STRESS INDUCED CELL DAMAGE AND DEATH

(71) Applicant: Redox Balance LLC, Danville, VA (US)

(72) Inventor: Stephen R. Wilson, Danville, VA (US)

(73) Assignee: Redox Balance LLC, Danville, VA (US)

(21) Appl. No.: 16/685,729

(22) Filed: Nov. 15, 2019

(51) Int. Cl. A61K 31/015; A61K 31/41; A61K 31/135; A61K 31/05; A61P 39/06

(52) U.S. Cl. CPC A61K 31/015; A61K 31/41; A61P 39/06; A61K 31/05; A61K 31/135

(57) ABSTRACT

Fullerene compounds and formulations are taught as useful in reducing oxidative stress in an organism because of their antioxidant properties and REDOX balance (homeostasis) properties.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a composition for the modulation of oxidative stress or redox signaling. In particular, it relates to fullerene compositions and related enzyme mimics which modulate oxidative stress and redox balance.

### Description of Related Art

Oxidative stress is a biological state that occurs when a cell's antioxidant capacity is overwhelmed by reactive oxygen species (ROS) causing a redox imbalance. Reactive oxygen species are a type of free radical, which is formed with oxygen. Free radicals are chemical species that contain one or more unpaired orbital electrons and are therefore unstable and liable to react with other molecules to form more stable compounds with a lower energy state. In an attempt to achieve this stable state, ROS can react with proteins, lipids, and DNA within the cell. This can result in cell damage and even cell death by inactivation of cellular components such as enzymes, membranes, and DNA. As such, ROS and oxidative stress as a whole have been suggested to participate in the initiation and/or propagation of diseases such as cardiovascular and inflammatory diseases, cancer, and diabetes by causing or exacerbating cell death.

ROS can be produced on a regular basis during oxidative metabolism and in more potent levels during inflammatory processes. It can also be present in administered products. During oxidative metabolism, electrons are lost from the electron transport chain and combine with oxygen, resulting in the formation of superoxide radical anions (O₂−.). At the time of inflammation, macrophages and neutrophils that contain the NADPH oxidase complex generate superoxide radicals and hydrogen peroxide to aid in the destruction of foreign agents. Environmental factors such as tobacco smoke, UV radiation and exposure to atmospheric oxygen, as well as overexertion during exercise and the consumption of alcohol and certain foods, can also result in the generation of too much ROS. Though many of these factors can be avoided or limited, as humans, our omnivorous diet exposes us to a variety of foods, some of which may contribute to increased oxidative stress in the gut. An uncontrolled increase of ROS in the gastrointestinal mucosa can lead to inflammatory or ischemic disorders. Oxidative stress has been postulated to play a role in inflammatory bowel disease (IBD) initiation and progression. The binding of an inflammatory stimulus to its cellular receptor triggers the activation of specific intracellular signaling pathways to upregulate the production of inflammatory mediators. Therefore, antioxidative stress mechanisms and antioxidants are key to limiting the proliferation of ROS and re-establishing a stable redox balance.

It would be useful to find compositions that could modulate/reduce oxidative stress or redox signaling in a cell to prevent damage or even death of cells (apoptosis) or an entire organism.

Biofullerene compositions are reported in detail in the Wilson 2000 U.S. Pat. No. 6,162,926, in the name of Murphy, Wilson, Lu, titled “Multisubstituted Fullerenes and Methods for Their Preparation and Characterization.” Biofullerene applications for neurodegenerative diseases have also been reported. Details of the use of fullerene additives in classical genomic experiments of transfection or transformation have been extensively described.

Both natural and artificial mixtures of C60 and C70 fullerenes exist. Fullerene concentration in carbon soot varies from parts per million in nature to much higher concentrations (1-14%) in soot from specially-designed manufacturing processes. This “as produced” fullerene soot is usually at least 70% C60 and about 25% C70. Other fullerenes such as C76, C78, and C84 are also present at less than 5% (see FIG. 1). In methods for rendering these compositions water soluble, the addition of groups to the fullerene is well known (FIG. 2). Addition of polar groups to the bucky ball involves, for example, addition of hydroxyl groups (polyhydroxy-C60), sulfate groups (FC4S) (see FIG. 3), or carboxyl groups (such as C3), (see FIG. 4).

### BRIEF SUMMARY OF THE INVENTION

The present invention relates to the discovery that a combination of C60 and C70 fullerenes in a formulation will reduce oxidative stress and modulate redox signaling. This prevents cell injury or cell death due to oxidative damage. Use of the compositions of the present invention allows for preventing cell damage and can therefore extend the life of the cell or organism once treated with the compositions of the present invention.

Accordingly, in one embodiment, there is a method of modulating oxidative stress or redox signaling in a living cell in need thereof comprising administering an effective fullerene composition which contains one of C60 or a C60/C70 mixture comprising at least about 70% C60 wherein the composition regulates at least one reactive oxygen species (ROS) and mimics superoxide dismutase (SOD).

In another embodiment, there is a process for the production of e,e,e-C60 fullerene tris-malonic acid (C3) comprising the steps of:
a. preparation of a tris-linker using 1,8-octanediol and malonic acid/dicyclohexylcarbodiimide at a concentration of 1-100 mmolar with ethyl acetate as the solvent;
b. reaction of macrocyclic trimer with C60 fullerene in toluene with iodine/1,8-diazabicyclo[5.4.0]undec-7-ene; and
c. purification of C3 from byproduct C3V by chromatography.

### BRIEF DESCRIPTION OF THE DRAWINGS

FIG. 1 shows the structures of C60 and C70, as well as other minor fullerenes C76, C78, and C84 which could be in the composition.

FIG. 2 shows a substituted fullerene.

FIG. 3 shows structures of two typical water-soluble C60 compounds of the present invention, polyhydroxy C60 and FC4S.

FIG. 4 is the structure of C3.

FIG. 5A is gamma cyclodextrin used to create the water-soluble 2:1 complex shown in FIG. 5B.

FIG. 6 is the process for the synthesis of C3 macrocyclic trimer intermediate.

FIG. 7 shows the manufacture of C3 utilizing macrocyclic trimer intermediate.

FIG. 8 shows the structure of cannabidiol (CBD).

FIG. 9A shows the structure of glutathione peroxidase mimic Ebselen. FIG. 9B shows the glutathione peroxidase mimic compound [2,2′-diselenobis-(N,N-dimethylamino)methylbenzene] bis(hydrochloride) salt.

FIG. 10A shows before result of sun-burned skin treatment with water soluble fullerene. FIG. 10B shows after result of sun-burned skin treatment with water soluble fullerene showing prevention of skin death and peeling.

FIG. 11A shows before treatment of wart with water-soluble C60 derivative FC4S (FIG. 3). FIG. 11B shows after treatment of wart with water-soluble C60 derivative FC4S (FIG. 3).

FIG. 12A shows water-soluble C60 treated tomato plants after two weeks. FIG. 12B shows water-soluble C60 untreated tomato plants after two weeks. Treated plants weighed 2× untreated plants.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### DEFINITIONS

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “X, Y, or Z” means any of the following: “X, Y, Z”; “X, Y”; “Y, Z”; “X, Z” etc. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “oxidative stress” refers to an imbalance between the production and introduction of reactive oxygen species (ROS) and scavenging systems of reactive oxygen species in the living body or cell (plant or animal) resulting in an excess of the reactive oxygen species. As this state worsens, nucleic acids, proteins, lipids, and the like—constituents of living cells—are oxidized resulting in damage to the cell or entire organism. Upon exposure to oxidative stress from internal or external sources, cells work toward a body's defense by inducing the expression of antioxidant proteins or phase II detoxification enzymes such as glutathione peroxidase. Thus oxidative, stress can damage cells and even cause cell death or cause the entire organism to die.

As used herein, the term “modulating” refers to modulation of “oxidative stress response” and refers to a physiological response in a biological sample (e.g., one or more cells in an organism) to an imbalance between the production of reactive oxygen species (ROS), chemically reactive molecules comprising oxygen ions including, but not limited to, superoxide anion (O₂−.), hydroxyl anion (.—OH), peroxynitrite (.OONO), nitric oxide (NO.), and hydrogen peroxide (H₂O₂). The cellular regulation of ROS through enzymes, antioxidants, and other compounds facilitate ROS breakdown and keep the cell in a state of redox balance, or homeostasis.

As used herein, the term “redox signaling” refers to regulated alterations in the intracellular redox state (redox signaling) and can modulate events such as DNA synthesis, enzyme activation, selective gene expression, regulation of cell cycle, cell growth, and programmed cell death.

Oxidative stress and resulting signaling pathways have been known for quite a long time but their detailed operational biochemistry is quite complex. A commercial agricultural product known as Harpin [C. Oh, et. al. 2007. “Growth-enhancing effect of HrpN in Arabidopsis”. Plant Physiol. 145: 426 (2007)] turns on genes that control redox homeostasis in plants. Knowledge of similar functionality in animals has lagged. Recently some data has emerged on animal redox homeostasis (C. Lipina, et. al., “Modulation of cellular redox homeostasis by the endocannabinoid system,” Open Biology 150276 (2016). The endocannabinoid ROS signaling system uses cannabidiol (FIG. 8 known as CBD) compounds and their receptors to control production of enzymes and regulatory proteins that have critical functions in normal cells. Evidence pertaining to CBD-induced regulation of ROS generation and scavenging mechanisms, systems cross-talk, and effects on propagation of abnormalities and pathologies are just beginning to emerge. Clearly, fullerene formulations of the invention work within this framework and provide additional beneficial effect.

As used herein, the term “living cell” refers to cells having an active metabolism, including cells capable of reproduction, wherein the cell is in a living organism. Death of a cell is often called apoptosis.

As used herein, the term “C60/C70 mixture” refers to mixtures of C60 and C70 fullerenes wherein there is at least about 70% C60 fullerene in the mixture and some portion of the rest is C70. It also, in one embodiment, contains other forms of fullerenes including C76, C78, and C84. The fullerenes are ball shaped fundamental forms of carbon that are formed whenever carbon is burned. Mixtures may be naturally occurring or mixed from individual component fullerenes. They can also be functionalized. For most biological applications (circulation in fluid or blood, entry into cells, and distribution through tissue) a water-soluble composition is usually desired. Modified versions of fullerenes which are both water soluble or non-water soluble are well known. Making the molecule polar makes it water soluble and making it non-polar makes it organic solvent or oil soluble. It is noted the compositions can be substituted or unsubstituted and be polar or non-polar, as desired. The key activity of this combination is an ROS scavenging antioxidant. This means its activity is like an enzyme in that it carries out a catalytic cycle by quenching the undesired oxidant and then is reduced by a cofactor, such as glutathione, back to its original state. This recycling effect allows the fullerenes of the present invention to be much more active per gram (up to 250 times) than other antioxidants such as Vitamin C.

As used herein, the term “reactive oxygen species” (ROS) refers to compositions which are oxidants in living cells and that includes, but are not limited to, hydrogen peroxide, hydroxyl radical, superoxide anion (O₂−.), singlet oxygen, nitric oxide, peroxyl radical, and peroxynitrite radical anion.

As used herein, the term “superoxide dismutase” (SOD) refers to an enzyme that scavenges superoxide anions having the formula of O₂−. In normal cells, when expression of SOD is lowered or eliminated, cells are triggered to die. Under these conditions of low SOD, the fullerenes of the present invention mimic SOD such that they prevent cells from dying by replacing any missing naturally occurring SOD.

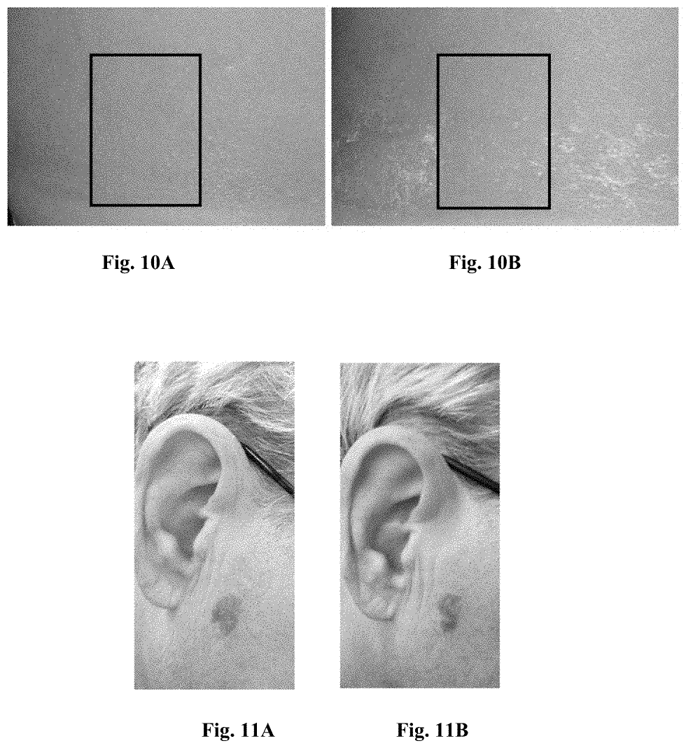

As used herein, the term “a glutathione peroxidase enzyme mimic” refers to compositions which have roughly the same activity profile as glutathione peroxidase. In one embodiment, the mimic is Ebselen (see FIG. 9A). Other glutathione peroxidase mimics include, for example, [2,2′-diselenobis-(N,N-dimethylamino)methylbenzene] bis-(hydrochloride) salt (see FIG. 9B). Both compounds are the subject of Spector, Wilson, Zucker, U.S. Pat. No. 5,321,138 (1994) “Compounds Having Glutathione Peroxdase Activity and Use.”

As used herein, the term “modulation of oxidative stress induced cell death” refers to both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side effects) resulting from administration of the treatment. On the other hand, the term “ineffective” indicates that a treatment does not provide a sufficient pharmacological effect to be therapeutically useful, even in the absence of deleterious effects, at least in the unstratified population. (However, a treatment may be deemed effective based on its effectiveness in the general population, even though it may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

The key observation of this disclosure is that fullerene compounds of the invention maintain redox balance or homeostasis by regulation of ROS in a manner similar to SOD. While fullerene compounds are super-antioxidants, the use of fullerenes for regulation of ROS is quite different from using massive doses of antioxidants such as vitamin C or E. Vitamin C and E can function as both antioxidants and prooxidants. The studies recently summarized [A. Gorlach, et. al., “Reactive oxygen species, nutrition, hypoxia, and diseases: Problems solved?” Redox Biology, 6, 372-385 (2015)] highlight treatments that illustrate misunderstanding REDOX balance and the cellular signaling modulation methods reported herein.

Oxidation-induced cell damage is undesirable in both disease states and general health. In animals, examples of disease states and general health conditions include, but are not limited to, oxidative stress induced muscle fatigue, muscle strain, injury or damage to cells, macular degeneration, cataracts, sunburn, hair issues (graying, alopecia), inflammation, psoriasis, itch, fertility issues (sperm and egg life), wound healing, ischemia, sepsis, reperfusion injury, anxiety, aging, and the like. Human disease effects also include Alzheimer's disease, ALS, Parkinson's disease, and diabetes, In plants, conditions affected by oxidative stress include general growth rate and plant health, prevention of leaf or needle drop, damage from freezing, insect damage, physical injury (as occurs in turf grass), and post-harvest spoilage. Controlling oxidative stress will improve production, as well as mitigate post-harvest spoilage of crops as well as valuable products such as essential oils and plant components such as CBD from cannabis.

Due to their enzyme mimic, super-antioxidant, and free radical scavenging properties, several fullerene C60 formulations improve plant health and growth. One such formulation uses fullerene soot, the charcoal created by combustion of carbon to produce fullerenes. This fullerene soot has beneficial properties for both plants and animals

It is well known that charcoal itself is beneficial for human health. Currently charcoal is being studied for additional medical applications and is a widely available health supplement. Charcoal has also been used to improve plant health. We have discovered that charcoal fullerene soot used as a soil additive, absorbs toxins, improves carbon and mineral composition, and enhances soil nutrients, which all serve to improve fertility and productivity, leading to increased yields and improved plant health for most crops.

In connection with the administration of a composition or drug, a drug which is “effective against” a disease or condition indicates that administration in a clinically appropriate manner results in a beneficial effect for at least a statistically significant fraction of patients, such as an improvement of symptoms, a cure, a reduction in disease signs or symptoms, extension of life, improvement in quality of life, or other effects generally recognized as positive by medical professionals familiar with treating the particular type of disease or condition.

As used herein, the term “fullerene” in its unsubstituted form refers to a compound composed entirely of carbon in the form of a hollow sphere or spheroid. Each carbon atom is connected by one double bond and two single bonds to other carbon atoms. Spherical fullerenes generally have a mixture of pentagonal faces and hexagonal faces, non-limiting examples which include C60, C70, C76, C78, C84 more-or-less spherical fullerenes. Fullerenes can be substituted or unsubstituted, especially to control solubility. They retain their antioxidant capacity when substituted and are included in the disclosure. Tubular fullerenes also exist and are called carbon nanotubes. They are not included in this disclosure.

As used herein, the term “C3” refers to e,e,e-C60 fullerene tris-malonic acid (see FIG. 4). A new process for making this composition is disclosed. As used herein, the term C3V refers to a minor isomeric by-product in the preparation of C3.

As used herein, the term “antioxidant” refers to a molecule capable of slowing or preventing the oxidation of other molecules. Oxidation is a chemical reaction that transfers electrons from an oxidizing agent to another substance. Such reactions can be promoted by and/or produce superoxide anions or peroxides. Oxidation reactions can produce free radicals, which start chain reactions that can damage cells. Antioxidants terminate these chain reactions by removing free radical intermediates and inhibit other oxidation reactions by being oxidized themselves. As a result, these “oxidized antioxidants” usually need reducing agents such as thiols, ascorbic acid, or polyphenols to reduce them back to their starting state. Antioxidants include, but are not limited to, α-tocopherol (vitamin E), ascorbic acid (vitamin C), porphyrin, α-lipoic acid, and n-acetylcysteine.

As used herein, the term “effective amount” refers to the amount of an agent to produce the intended pharmacological, therapeutic, or preventive result. The pharmacologically effective amount results in the amelioration of one or more signs or symptoms of a disease or condition, or causes a halt in the progression or the regression of the disease or condition. For example, a therapeutically effective amount refers to the amount of a therapeutic agent that increases cell life or reduces cell damage from oxidants. The effective amount can be given in single or multiple daily doses (or less frequently) sufficient to cause the intended effect.

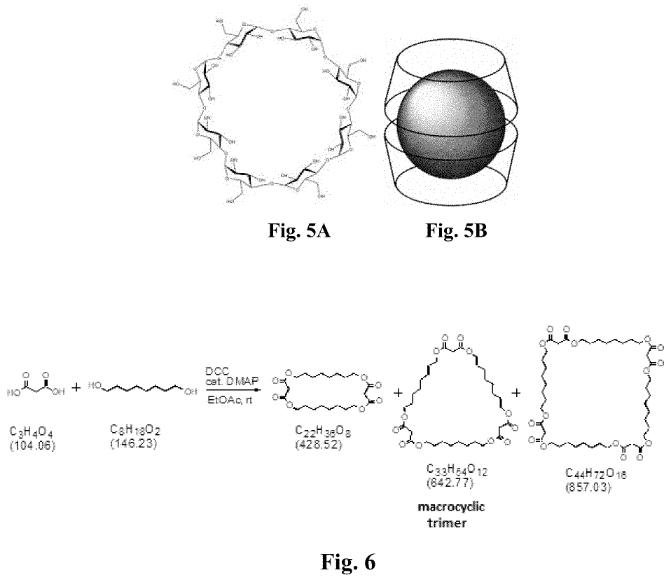

As used herein, the term “polar” refers to compositions which are water soluble. Such compounds and methods to make them are well known. In one embodiment, cyclodextrin (See FIG. 5A) is used to create a complex with C60 (FIG. 5B) thus making the complex water soluble.

As used herein, “non-polar” refers to fullerenes that are not soluble in water. For use in cells or any living organism (animal or plant), fullerenes can also be dissolved in an oil. Products on the market that contain only C60 are normally dissolved in olive oil. It has been discovered that the amount of polyunsaturated fatty acids is critical in optimizing solubilization. In one embodiment, an edible oil is selected for dissolution of the fullerenes of the present invention. That oil has, at a minimum, 50% polyunsaturated fatty acids. Oils such as corn, cottonseed, flaxseed, hempseed, and soybean all meet this criteria. In one embodiment, the oil is hemp oil. Compositions are formulated with appropriate excipient additives and the like, as well as the edible oils, for administration.

New process chemistry for C3 preparation has taken the original process and changed and optimized the chemistry, reaction conditions, materials, and solvents to a workable and scalable manufacturing process. The critical step (and worst step in the old process) was the preparation of a macrocyclic linker. Major fundamental improvements were (1) to change from malonyl dichloride to malonic acid, (2) to change from methylene chloride to ethyl acetate as solvent, and (3) the initial reaction conditions were made 5 times (5×) more concentrated (leading to potential savings in solvent and processing costs). The final compound of the present invention is the compound called C3 (See FIG. 4), which is a very well tolerated water soluble fullerene.

### EXAMPLE 1—PREPARATION OF C3

A 5 L, 3-necked R.B. flask with a mechanical stirrer, N2 flow adapter, and a glass stopper, were charged with 1,8-octanediol (14.6 g, 0.10 mol), malonic acid (10.4 g, 0.10 mol), and EtOAc (3 L). The mixture was stirred for 0.5 hrs at room temperature until all the solids were dissolved. To this solution, dicyclohexylcarbodiimide (DCC , 41.3 g, 0.20 mol) and dimethylaminopyridine (DMAP, 3.67 g, 0.03 mol) were added and the resulting white suspension was stirred for 24 hrs at room temperature. The reaction mixture was concentrated to about one-half volume at reduced pressure, then filtered. The filter cake was washed with ethyl acetate (2×300 ml). The filtrate was concentrated under reduced pressure to a small volume. The residue was slurried in 1:1 toluene:hexanes (ca. 65 ml) and the resulting solids (mostly pure dimer) were filtered off and the filtrate was concentrated. The residue was purified by flash column chromatography (150 g, 5.5×15 cm), using a gradient elution starting with hexane (0.5 L), then 10% EtOAc/hexane (1 L), then 20% EtOAc/hexane (2.0 L). The product eluted with 20% EtOAc/hexane. Yield 1.44 gm (6.7%). Reaction of C60 with macrocyclic trimer (cyclo-[3]-octylmalonate): Synthesis of the e,e,e-(C3-precursor) and trans-4, trans-4, trans-4-(C3V-precursor) trisadducts in a dry 3-necked flask equipped with gas inlet, 250 ml dropping funnel and magnetic stirrer, 255 mg (0.354 mmol/1.0 eq.) C60 were dissolved under argon in 400 ml of dry toluene. Subsequently, 205 mg (0.319 mmol/0.9 eq.) of the macrocycle and 243 mg (0.956 mmol/2.7 eq.) of iodine were added to the solution. Then, a solution of 404 mg (397 μl/2.65 mmol/7.5 eq.) DBU (1,8-diazabicyclo[5.4.0]undec-7-ene) in 160 ml of dry toluene was added dropwise over a period of 3 hours. The color of the solution turned to a deep orange. After additional stirring at room temperature for about 10 mins, the crude mixture was subjected to flash chromatography on silica gel (6×25 cm). Traces of C60 and other impurities were eluted with toluene, then the eluant was changed to toluene/ethylacetate 98:2 and the trisadducts C3-precursor and C3V-precursor were eluted together as a bright orange band. C3-precursor and C3V-precursor have been separated by preparative HPLC on Nucleosil (toluene/ethylacetate 98:2). The product fractions were evaporated, precipitated from CH2Cl2/pentane, washed three times with pentane, and dried at 60° C. in high vacuum.

### EXAMPLE 2. SPRAY FOR HAIR

A formulation of a water soluble fullerene compound such as polyhydroxy-C60 or FC4S (FIG. 3) 0.6 mg/mL can be used to rejuvenate hair by spraying the compound on the hair and exposed scalp. For healthy individuals with an adequate diet and nutrients, hair will be noticeably improved within a few months of daily treatment. The texture of the hair improves and becomes more manageable; it also appears more lustrous. For those individuals whose hair has recently begun to thin and turn gray, new hair growth can occur. Often the new hair growth is the original hair color. With continued use of the compound, existing gray hair may also regain its original hair color, while the condition and appearance of the hair continues to improve. Over time, hair is visibly thicker; and gradually the head of hair more closely resembles its original density, color, condition, and luster.

### EXAMPLE 3. HEMP OIL SKIN CREAM

Formulation of C60 in Hemp oil (˜1 g/liter) can be used as a skin cream for treatment of skin sores, abrasions, cuts, and even blemishes. One example uses the C60 skin treatment for preventing skin damage (peeling) after sunburn. Before and after results are shown in FIGS. 10A and 10B. Another example uses either water or oil soluble C60 for removal of warts. After topical application once or twice a day for several months, large warts that have been growing for years start to recede and gradually disappear. Another formulation which was used was water-soluble C60 in Aloe Vera. FIG. 11 shows before (A) and after (B) 3 months treatment of a large wart.

### EXAMPLE 4. HUMAN HEALTH SUPPLEMENT

Formulation of water soluble C60 compound polyhydroxy-C60 or FC4S (FIG. 3) can be used as a health supplement for humans to improve health. As a super-antioxidant (˜250 times stronger than Vitamin C) fullerene redox balance action removes free radicals and stabilizes the life-death signaling which results in diverse health benefits. Typical formulation is 0.6-1 gram per gallon of water with a daily dose of 20-100 mL. Formulation of C60 in hemp oil can also be used but usually at a higher dose (˜1 gram per liter). In addition, our other antioxidant enzyme mimic Ebselen (FIG. 9A) or [2,2′-diselenobis-(N,N-dimethylamino)methylbenzene] bis(hydrochloride) salt (FIG. 9B) can be added at ˜⅕ the C60 dose. Ebselen was previously patented for topical skin applications (Natterman, EU Patent No.: 87106773.2, Aug. 5, 1987). It is also appropriate to add other antioxidants and/or vitamins to the formulation of this invention. An additional health supplement formulation uses charcoal fullerene soot to combine the health supplement benefits of charcoal with super-antioxidant redox balance benefits. Typical formulation of charcoal fullerene soot may add about 2% fullerene to charcoal. Additionally, the typical as-produced fullerene soot from the manufacturing of fullerenes, which is charcoal usually containing 1-14% fullerene, can be used.

Numerous health benefits result from all of these regimens and the formulations could also be employed for more serious conditions such as Parkinson's, ALS, or Alzheimer's.

### EXAMPLE 5. FULLERENE-CBD HEALTH SUPPLEMENT

Formulation of C60/C70 natural mixed fullerene or other fullerenes can be used in combination with cannabidiol (FIG. 8, CBD) in oil or water. Taken orally, the supplement can relieve pain, boosts overall health, and improves a number of medical conditions. The combination of fullerenes with CBD works synergistically with redox balance effects thus being more effective at improving health and medical conditions than CBD alone.

### EXAMPLE 6. PLANT ENHANCER

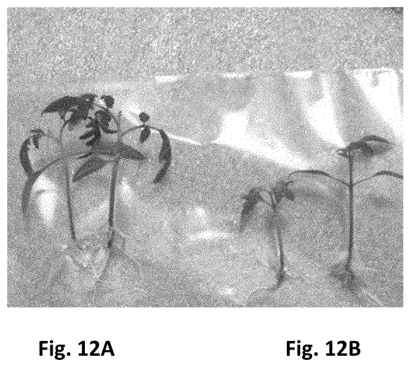

Formulation of water soluble C60 compound polyhydroxy-C60 (FIG. 3) can be used for seed pre-treatment to enhance germination or to treat plants to enhance growth. Soaking seeds for 1 hour prior to placing in growing medium leads to significant enhancement of germination. Also, treatment of clones before initial planting leads to increases in growth rate—up to two times. Results are shown in FIGS. 12A and 12B. The same overall plant growth enhancement can additionally be achieved by foliar spray of crops in the field with water-soluble fullerenes at ˜2-5 g/acre. We have also found that after harvest, previously treated crops appear to stay fresh longer.

### EXAMPLE 7. FULLERENE SOOT FOR SOIL AND PLANT ENHANCEMENT

For plants, fullerene soot is a soil additive utilizing the combination of effects of carbon soot in soil composition and fullerene super-antioxidant activity that results in faster growing and stronger plants. A new discovery has been that there are positive plant enhancement effects of fullerenes present in the fullerene soot—the charcoal created by combustion of carbon in the manufacturing process for fullerenes. While charcoal itself is widely used in agriculture and is known to have a beneficial effect on soil fertility and productivity, we now reveal that “charcoal fullerene soot” combines the considerable benefits of charcoal as a soil nutrient with the super-antioxidant plant health effect of fullerene to result in an increase in yields for most crops. One typical formulation is fullerene soot charcoal containing 1-14% fullerenes with the remainder typical amorphous carbon. A soil enhancement treatment involves amendment of the top few inches of soil to ˜5% charcoal. This amended layer absorbs toxins, improves available carbon nutrient content and both the soot and the fullerenes produce their redox balance effect on the growing plants.

### DRAWINGS

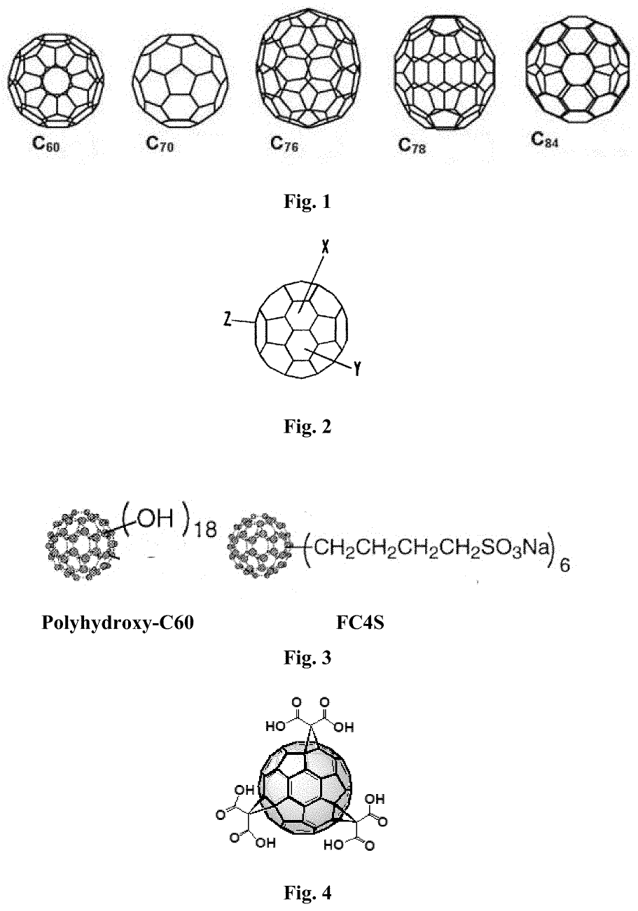

Now referring to the drawings, FIG. 1 shows the structures of the fullerenes of the present invention. Shown from left to right is C60, C70, C76, C78, and C84. While shown unsubstituted, these compositions can be substituted and made either polar or non-polar. In one embodiment, C60 and C70 make up 95% of all fullerenes in the composition.

FIG. 2 is an example of a fullerene with 3 substitutions. Obviously more or fewer additions could be utilized.

FIG. 3 shows the structure of two water-soluble substituted C60 compositions of the present invention (polyhydroxyl-C60 and FC4S).

FIG. 4 shows a particular C60 composition named C3.

FIG. 5A shows gamma-cyclodextrin (commercial Captisol®) which is used to make the water-soluble 2 to 1 (binary) complex shown in FIG. 5B.

FIG. 6 is the process of producing the C3 macrocyclic trimer intermediate used for C3 synthesis.

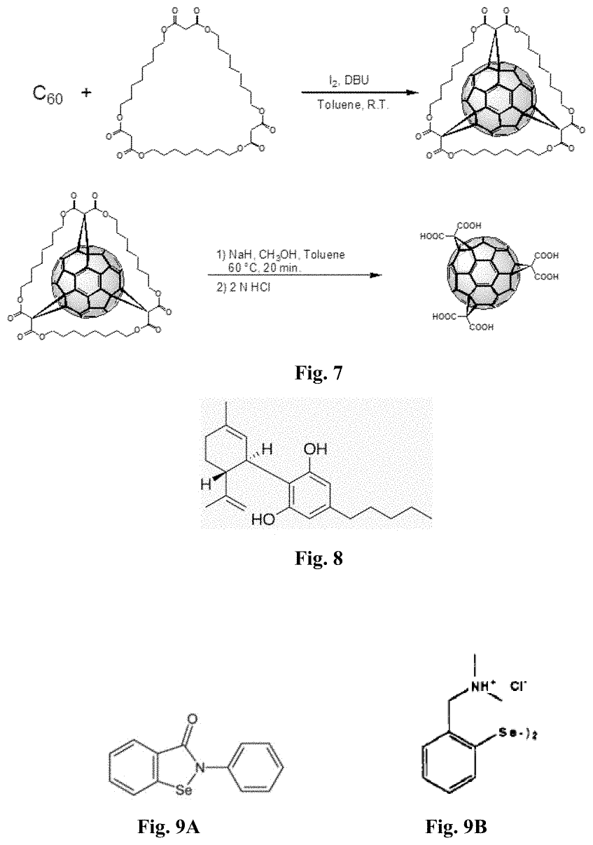

FIG. 7 shows the manufacture of C3 utilizing C3 macrocyclic trimer intermediate.

FIG. 8 shows the structure of cannabidiol (CBD).

FIG. 9A shows the structure of glutathione peroxidase mimic Ebselen. FIG. 9B shows the glutathione peroxidase mimic [2,2′-diselenobis]-(N,N-dimethylamino)methylbenzene] bis(hydrochloride) salt.

FIG. 10A shows before result of sun-burned skin treatment with water soluble fullerene. FIG. 10B shows after result of sun-burned skin treatment with water soluble fullerene showing prevention of skin death and peeling.

FIG. 11A shows a large wart before treatment. FIG. 11B shows wart shrinking in size after treatment of wart with water-soluble C60 derivative (See FIG. 3).

FIG. 12A shows water-soluble C60 treated tomato plants after two weeks. FIG. 12B shows water-soluble C60 untreated tomato plants after two weeks. Treated plants weighed 2× untreated plants.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, other modifications of structure, sequence, materials, and the like, apparent to those skilled in the art, still fall within the scope of the invention as claimed by the applicant.

What is claimed is:

1. A method of modulating oxidative stress or redox signaling in a living cell in need thereof comprising administering an effective fullerene composition which contains one of C60 or a C60/C70 mixture comprising about 70% C60 wherein the composition regulates at least one reactive oxygen species (ROS) and mimics superoxide dismutase (SOD).

2. The method according to claim 1 wherein the composition comprises a charcoal fullerene soot which contains 1-14% mixture of fullerenes.

3. The method according to claim 1 wherein the composition further comprises the addition of a glutathione peroxidase enzyme mimic.

4. The method according to claim 3 wherein the glutathione peroxidase enzyme mimic is selected from the group consisting of Ebselen and [2,2′-diselenobis-(N,N-dimethylamino)methylbenzene] bis(hydrochloride) salt.

5. The method according to claims 1 to 4 wherein sufficient composition is utilized to prevent oxidative stress induced cell death in a living cell of an animal.

6. The method according to claims 1 to 4 wherein sufficient composition is utilized to prevent oxidative stress induced cell death in a living cell of a plant.

7. The method according to claims 1 to 4 which further comprises at least one additional antioxidant.

8. The method according to claims 1 to 4 which further comprises at least one additional ROS regulator.

9. The method according to claims 1 to 4 which further comprises addition of cannabidiol compounds such as CBD.

10. The method according to claims 1-4 wherein the fullerene in the composition is water soluble.

11. The method according to claims 1-4 wherein the fullerene is functionalized.

12. The method according to claims 1-4 wherein the fullerene composition is polar and is dissolved in water.

13. The method according to claims 1-4 wherein the fullerene composition is non-polar and is dissolved in an edible oil that is at least 50% polyunsaturated.

14. A process for the production of e,e,e-C60 fullerene tris-malonic acid (C3) comprising the steps of:
a) preparation of a tris-linker using 1,8-octanediol and malonic acid/dicyclohexylcarbodiimide at a concentration of 1 to 100 mmolar with ethyl acetate as the solvent;
b) reaction of macrocycle trimer with C60 fullerene in toluene with iodine/1,8-diazabicyclo[5.4.0]undec-7-ene; and
c) purification of C3 from byproduct C3V by chromatography.
